(19) United States
(12) Patent Application Publication
(10) Pub. No.: US 2012/0095093 A1
(43) Pub. Date: Apr. 19, 2012

(54) ANTIFUNGAL COMPOUNDS

(76) Inventors: William E. Courchesne, Soda Springs, CA (US); Elzbieta Hejchman, Warsaw (PL); Dorota Maciejewska, Warsaw (PL); Jerzy Kossakowski, Warsaw (PL); Kinga Ostrowska, Warsaw (PL)

(21) Appl. No.: 13/330,349

(22) Filed: Dec. 19, 2011

Related U.S. Application Data
(63) Continuation of application No. 12/111,433, filed on Apr. 29, 2008.
(60) Provisional application No. 60/915,826, filed on May 3, 2007.

(51) Int. Cl. A61K 31/343; A01P 3/00; A61P 31/10; C07D 307/86; C07D 307/85; A01N 43/12

(52) U.S. Cl. 514/469; 549/468

(57) ABSTRACT

Antifungal compounds having a benzofuran structure. Antifungals exhibit much less toxicity to human cells than amiodarone. These compounds are useful alone or in combination with other compounds having antifungal activity. Compounds of this invention exhibit synergistic antifungal activity in combination with antifungal amiodarone compounds. The invention provides pharmaceutical compositions useful for treating fungal infections, methods for treating fungal infections and compounds useful in such compositions for treating fungal infections. Compounds of this invention are further useful as antifungal agents for the prevention and or treatment of fungal infections in plants. The invention includes agriculturally useful compositions comprising one or more compounds of this invention which exhibit fungistatic or fungicidal function against one or more plant pathogen is a fungus.

### CROSS-REFERENCE TO RELATED APPLICATIONS

This application takes priority form U.S. provisional application 60/915,826 which is incorporated by reference herein in its entirety.

### BACKGROUND OF THE INVENTION

The present invention relates generally to methods of treating fungal infections and methods of killing or inhibiting growth of fungi. Pathogenic fungi occur world wide and are major agricultural and health pests. Fungal infections in humans range from superficial and cutaneous to deeply invasive and disseminated. Treatment of fungal infections has lagged behind bacterial chemotherapy. There are substantially fewer antifungal drugs than antibacterial drugs.

The incidence of fungal infections has risen dramatically due to an increase in the number of people with AIDS, undergoing bone-marrow and solid organ transplantations, high-dose chemotherapy, steroid treatment, and invasive medical procedures, moreover, treatment options are limited (Bodey et al., 1992; Groll and Walsh, 2001; Marr et al., 2002; 1999; Pfaller et al., 1998; Sussman et al., 2004). Fungal opportunistic infections such as candidiasis, cryptococcosis, and histoplasmosis, occur frequently in patients with AIDS. Among the opportunistic infections, fungal infections caused by Pneumocystis, Candida, Cryptococcus, or Histoplasma were the first to occur in more than 50% of persons with AIDS; and at time of death, nearly 85% of decedents had a fungal infection (Jones, et al., 1999).

Cryptococcus neoformans is an opportunistic yeast pathogen that can cause cryptococcosis, a lethal meningitis in individuals with compromised immune systems (Casadevall and Perfect, 1998.) Cryptococcosis is most common in patients with defects in cellular immunity and thus occurs in association with AIDS, transplantation, steroid treatment, and lymphoma ((Jones et al., 1999; Kwon-Chung and Bennett, 1992; Kwon-Chung et al., 2000; Mitchell and Perfect, 1995; Stansell, 1993; Sugar, 1991).

Although the incidence of cryptococcosis declined in the mid-1990s due to effective antiretroviral therapy and the prophylactic use of antifungals, in particular fluconazole, Cryptococcus is still a common cause of life-threatening infections.

Stem cell and solid organ transplantations have increased dramatically in recent years and transplant patients are some of the most significant immunosuppressed individuals at risk for invasive aspergillosis (Singh and Paterson, 2005.) The frequency of Aspergillus infections is generally around 20% in hematopoietic stem cell transplant (HSCT) recipients and can be as high as 15% in organ transplant patients. The mortality rate in transplant recipients with invasive aspergillosis is quite high, ranging from 74 to 92% and accounts for about 9 to 17% of all transplant recipients deaths in the first year. Overall, the last decade has seen a rising frequency of aspergillosis in large tertiary care centers.

There is an increasing frequency of Aspergillus species other than A. fumigatus isolated from HSCT patients. For example, A. terreus is the most common Aspergillus species that is detectable in the bloodstream and accounts for about 3% of the Aspergillus infections overall. For the years 1996 to 1998, 33.7% of positive cultures from HSCT recipients yielded non-A. fumigatus species, up significantly from 18.3% for the preceding 3 years. These data are of concern because A. terreus and some other fungi are innately resistant to amphotericin B, the major therapeutic for aspergillosis. Neutropenia has traditionally been the predominant risk factor, with most infections occurring prior to engraftment.

The licensed antifungals used systemically can be classified into 5 categories according to the targets of their action. The majority of these antifungals target ergosterol synthesis (azoles) or function (amphotericin B). The azoles act by inhibiting an enzyme, lanosterol demethylase, which participates in the synthesis of ergosterol, an essential component of fungal membranes. Azole antifungal drugs include, among others, clotrimazole, ketaconazole, fluconazole, and itraconazole (Graybill, 1996). Other commonly used drugs include flucytosine and amphotericin B. Amphotericin B has been the mainstay of antifungal therapeutics for many years, but is limited due to toxicity (nephrotoxicity and hypoxia) and the need to administer it intravenously, which is costly, tedious and associated with infusion-related problems. Lipid formulations of amphotericin B have reduced, but not eliminated, side effects, but must still be given intravenously. In addition, their cost is greater than an order of magnitude higher than traditional amphotericin B. The azoles, such as fluconazole can be given orally but are not effective against some fungi, such as Aspergillus and certain non-albicans Candida species. In addition, over the past decade, the wide use of azoles has also resulted in the appearance of resistant mycotic species and many of the emerging fungal pathogens are resistant to the currently used antifungal agents. Finally, most of the commonly used therapeutics are fungistatic and rely on the host's immune system for clearing the growth-arrested cells, which is a problem in immunocompromised patients.

Because of the increase in resistance to antifungals currently in use, there is a need to develop new fungicidal agents with novel mechanisms of action (Bastert et al., 2001; DiDomenico, 1999; Fostel and Lartey, 2000; Georgopapadakou and Walsh, 1996; Graybill, 1996). In particular, effective antifungal therapy for systemic mycoses is limited. Products and methods responsive to this need would ideally involve lower toxicity compounds available in large quantities. Ideal compounds would have a rapid effect and a broad spectrum of fungicidal or fungistatic activity against a variety of different fungal species when administered or applied as the sole antifungal agent. Ideal compounds would also be useful in combinative therapies with other antifungal agents, particularly where these activities would reduce the amount of antifungal agent required for therapeutic effectiveness, enhance the effect of such agents, or limit potentially toxic responses and high cost of treatment. Particularly advantageous would be compounds that are orally available and active for administration of antifungal agents.

Currently, roughly 10,000 fungal species are thought to be pathogenic for plants, however, that number is likely to increase (Agrios, 1997) since the number of fungal species now identified (estimated to be nearly 100,000) is only about 7% of the total number of fungal species (estimated to be about 1.5 million species) (Hawksworth, 2001).

Crop losses due to diseases caused by fungi pose a serious threat to the global supplies of food (for human and agricultural consumption) and fiber. Furthermore, mycotoxins are produced by certain groups of fungi (e.g., including but not limited to members of the genus Fusarium) during infection of crops, creating a significant health hazard to livestock, pets, and humans.

Novel and effective control of fungal pathogens causing diseases of fruit, vegetable, and ornamental crops is needed. The agricultural sector, like the medical sector, has been confronted with the occurrence of pathogens resistant to commercially available antifungals. Effective control of resistant pathogens has been problematic and expensive and has been complicated by the loss of some fungicides for use on some crops. Thus, it is especially desirable to use novel antimicrobial agents with unique activities that lack cross-resistance to the current repertoire of antifungals.

A number of plant diseases, including among others root rot, stalk rot, crown rot and damping off, are associated with fungal infection. Plant pathogen fungi include, among others, species of Pithium, Phytophthora (e.g., Phytophthora infestans), Fusarium, Rhizoctona (e.g., Rhizoctona solani), Thielaviopsis, Sclerotinia, Cylindrocladium, Gibberella, Colletotrichium and Aspergillus (e.g., Aspergillus flavus). Antifungal agents useful against one or more of such plant pathogenic fungi will be generally beneficial for treatment and prevention of plant fungal infection

Recently, amiodarone (Courchesne, 2002) was identified as an antifungal agent. See U.S. Pat. No. 6,221,903. Amidarone having structure A:

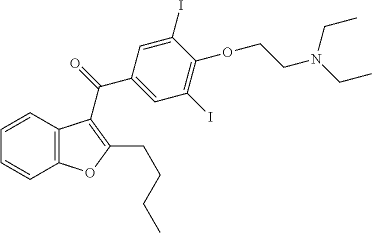

was developed over 30 years ago as an antianginal, but is now widely used as a potent antiarrhythmic drug (Gill et al., 1992; Mason, 1987; Roden, 1996; Singh, 1996). The hydrochloride salt and other salts of amiodarone have been used for treatment of cardiac arrhythmias. Because amiodarone is currently in clinical use, in theory, it could be used as an antifungal therapeutic. A significant consideration for clinical use of amiodarone is its side effects, including pulmonary toxicity. Compounds structurally related to amiodarone which retain antifungal activity, but which exhibit reduced toxicity toward mammalian cells, would provide significant benefit as antifungal agents. This invention relates to certain benzofuran compounds which exhibit antifungal activity, but which are significantly less toxic to mammalian cells than amiodarone.

### SUMMARY OF THE INVENTION

The present invention provides methods of treating a subject suffering from a fungal infection by administering a therapeutically effective amount or combined amount of a compound of formula I (see below). One or more compounds of this invention may be administered alone or in conjunction with known antifungal agents, including one or more amiodarone compounds, such as those described herein. When employed in adjunctive therapy, the administration of one or more compounds of this invention can reduce the amount of another antifungal agent, including amiodarone, needed for effective therapy, thus limiting potential toxic response and/or high cost of treatment. Co-administration of one or more compounds of this invention may also enhance or accelerate the effect of such antifungal agents.

The invention is also directed to novel compounds of formula I and to pharmaceutical compositions comprising a pharmaceutically acceptable excipient or carrier and one or more compounds of this invention of Formula I. The invention also provides a method of killing or inhibiting growth of fungi comprising contacting the fungi with one or more compounds of this invention (Formula I). This method can be practiced in vivo or in a variety of in vitro environments, such as, for decontamination of fluids and surfaces and for sterilization of surgical and other medical equipment and implantable devices, including prosthetic joints and indwelling invasive devices.

The compounds of the invention are also useful in the manufacture of a medicament for treatment of fungal infection. The medicament may include, in addition to the one or more compounds of this invention, other chemotherapeutic agents such as antifungal agents, particularly an amiodarone compound. The medicament may also include a pharmaceutically acceptable excipient or carrier appropriate for administration of the active ingredients therein.

The invention further provides methods of prophylactically or therapeutically treating an immunocompromised subject comprising the step of administering to said subject an amount of amiodarone compound effective to kill or inhibit replication of fungi.

The antifungal compounds and pharmaceutical compositions of this invention are useful for the treatment of fungal infection in animals other than humans, in particular in canine, feline, equine, and bovine species among others.

The invention also provides agriculturally useful formulations for application to plants exhibiting one or more symptoms of a fungal infection or to prevent fungal infections in plants or plant parts believed at the time of treatment to be free of such infection. Such formulations include one or more compounds of this invention of Formula I in an agriculturally acceptable carrier. Such agriculturally useful formulations include aqueous solutions and suspension as well as emulsions for spraying and dry formulations for dusting. Formulations can be administered by foliar application or by treatment of soil in the vicinity of plants or in which plants or seeds are to be grown.

Agriculturally useful formulations can be employed for treatment of whole plants, various plant parts (roots, stems, leaves, etc.) or seeds. Agriculturally useful formulations of this invention can be used as prophylatic formulations to prevent fungal inventions, for example, formulations can be applied to seed on planting or roots on transplanting. Additional agriculturally useful formulations of this invention can further be employed to treat harvested plants or plant parts to prevent or reduce infestation by fungal species, particularly those that are plant pathogens.

Additional aspects and advantages of the invention will become apparent to those skilled the art upon considering the following detailed description of the invention.

### BRIEF DESCRIPTION OF THE DRAWINGS

FIGS. 1A and 1B show the growth of a mammalian cell line, K-562 in the absence and presence of AR3. The increase in cell number was followed over time. The increase in cell number of cultures containing AR3 was found to be similar to the increase in control cells that were treated with the carrier, DMSO, only. The results demonstrate that AR3 shows no negative effects on growth of these mammalian cells at concentrations (10-30 μM) that are completely inhibitory to yeast growth.

FIGS. 2A and 2B show the effects of amiodarone and AR3 on the rise in cytoplasmic calcium ions. While amiodarone exposure causes an immediate and huge increase in cytoplasmic calcium ions, treatment with AR3 alone has no effect.

FIG. 3 shows the effect of pretreating yeast cells with AR3 prior to addition of amiodarone on the rise in cytoplasmic calcium ions. Although AR3 does not itself elicit a calcium mobilization, it does significantly potentiate the rise in cytoplasmic calcium caused by amiodarone treatment, demonstrating a synergistic effect of the combination of AR3 with amiodarone.

### DETAILED DESCRIPTION OF THE INVENTION

Compounds herein have antifungal activities, including fungicidal activity. Tests for antifungal activity of any compounds of this invention can, for example, be performed in in vitro killing assays and/or in in vivo models of fungal infection, as measured, for example, by improved survival or reduction of colony-forming units in circulation after fungal challenge including infections caused by Cryptococcus, such as cryptococcal meningitis, infections caused by Aspergillus, and infections caused by Candida species, including mucocutaneous and systemic candidiasis, may be treated according to the invention. As further described herein, antifungal activity of compounds of this invention can be assessed by assessing their effect on cytoplasmic Ca²⁺ levels. Assessment of the effect of a given test compound on cytoplasmic Ca²⁺ levels can be employed in a screen for antifungal activity.

Compounds of this invention include those having the structure:

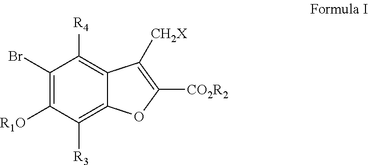

or a pharmaceutically acceptable salt thereof, wherein R₁, R₂ and R₄ are H, or an alkyl having 1-6 carbon atoms, where the alkyl group is optionally substituted with one or more halogens, hydroxyl groups, amino groups or a combination thereof; or one or both of R₁ and R₂ represent negative charges on the oxygen, which result in mono and disalts of Formula I; R₁ may also be an alkyl sulfonate or an aryl sulfonate, wherein the alkyl or aryl group is optionally substituted; R₃ is selected from the group consisting of H, alkyl, —CO—R, —OCO—R, —NR′CO—R, —CO—N(R)₂, and —CO₂—R, where each R and R′ are independently selected from H or an alkyl group having from 1 to 6 carbon atoms, wherein the alkyl group is optionally substituted with one or more halogens, OH, amino groups or a combination thereof; and X is selected from the group consisting of H, halogen, OH, alkylsulfonate, arylsulfonate, and an amino group.

In specific embodiments, the compounds of the invention include those of Formula I with the exceptions that R₃ and R₄ are not both H and that R₃ is a group other than H, when X is Br.

In other specific embodiments, the invention provides compounds of Formula I wherein:

R₁ is an unsubstituted alkyl group;
R₂ is an unsubstituted alkyl group;
R₄ is an unsubstituted alkyl group;
R₁ is a methyl or ethyl group;
R₂ is a methyl or ethyl group;
R₄ is a methyl or ethyl group;

### R₃ is H;

R₃ is an alkyl group;
R₃ is a methyl or ethyl group;
R₃ is an —CO—R group;
R₃ is an —CO—CH₃ group;
R₃ is an —CO—C₂H₅ group;
R₃ is an —OCO—R group or —CO₂—R group;
R₃ is an —NR′CO—R or —CO—N(R)₂, group;
each R and R′ are independently hydrogens or alkyl groups having 1-6 carbon atoms;
each R or R′ are independently alkyl groups substituted with one or more halogens;
each R or R′ are independently alkyl groups substituted with one or more OH groups;
each R or R′ are independently alkyl groups substituted with one or more amino groups;

### X is Br;

### X is OH;

### X is NH₂;

X is alkylsulfonate,
X is an alkyl sulfonate in which the alkyl group has 1-6 carbon atoms;
X is methyl sulfonate;
X is an aryl sulfonate; and
X is a phenylsulfonate.

The compounds of this invention include those having any one or more of the above listed combinations of variables that are stoichiometrically possible.

In specific embodiments, compounds of the invention include those of Formula I with the exception of the compounds where (1) R₁, R₂, R₄ and X are all hydrogens and R₃ is —COCH₃ and (2) R₂, R₃, R₄ are all hydrogens, X is Br, and R₁ is CH₃. In other specific embodiments, compounds of the invention include those of Formula I in which R₄ and X are both H; in which R₃ is —COCH₃, or in which R₃ is —COR, where R is an alkyl having 1-3 carbon atoms.

Compounds of this invention of Formula I can be prepared by methods described herein and in view of methods of synthesis which are well-known in the art. Kossakowski et al., 2005; Salvi and Sethna, 1967 and Shah and Shah, 1960 provide methods of synthesis of certain 2- and 3-benzo[b]furan carboxylic acids. Each of these reference is incorporated by reference herein for its description of synthetic methods and for its description of benzofuran compounds and derivatives which may be known in the art.

Treatment, as used herein, encompasses both prophylactic and therapeutic treatment. The methods herein involve treatment of an individual known or suspected to have a fungal infection and is meant to refer to higher organisms, including animals (e.g., humans; companion animals such as dogs; livestock such as horses, cows and pigs; poultry; insects; fish; avian species).

In related aspects, the compounds of this invention are useful in agricultural applications for treating fungal infections in plants.

Compounds of this invention includes pharmaceutically acceptable salts thereof.

The invention further relates to the combination of one or more compounds of this invention of Formula I, including pharmaceutically acceptable salts thereof, with an antifungal compound and particularly with an amiodarone compound for the treatment of fungal infections as described above.

An amiodarone compound, as used herein, refers to the agent designated amiodarone:

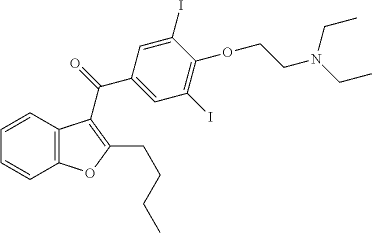

and specifically includes the salt formulation amiodarone hydrochloride {(2-Butyl-3-benzofuranyl) [4-[2-(diethylamino)-ethoxyl]-3,5-diiodophenyl] ketone, hydrochloride} known and marketed in the U.S. as Cordarone (Trademark, Sanofi-Aventis Corp., Paris, FR). An amiodarone compound, as used herein, also includes an amiodarone derivative that has antifungal activity according to the invention. Amiodarone derivatives for treatment of cardiac arrhythmias have been described (see, e.g., U.S. Pat. No. 5,849,788).

In specific embodiments, amiodarone compounds include those of Formula X which exhibit antifungal activity:

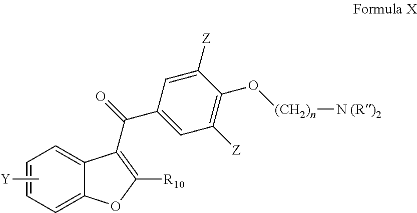

and pharmaceutically acceptable salts thereof including those in which the nitrogen is protonated or alkylated (—N(R″)₂H⁺ or —N(R″)₃⁺);
where each Z independently of one another is, I or H,
Y represents substitution with one or more substituent groups;
n is an integer ranging from 1 to 6;
R₁₀ is an unsubstituted alkyl group having 1 to 6 carbon atoms and
each R″, independent of other R″, is an alkyl group, particularly an alkyl group having 1-6 carbon atoms.

Formula X includes compounds in which n is 2-4, R″ are ethyl groups, R₁₀ is an ethyl, propyl butyl or pentyl group; or Y is hydrogen or an alkylsulfonate group.

Amiodarone compounds also include additional analogs and metabolites of amiodarone such as those described in Quaglin, D. et al., 2004. Amiodarone compounds specifically include, among others, dronedarone and desethylamiodarone and salts thereof.

Fungi are eukaryotic cells that may reproduce sexually or asexually and may be biphasic, with one form in nature and a different form in the infected host. The term fungus or fungi is used herein as broadly as the term is used in the art. The term fungus/fungi includes among many others various genus and species of yeast.

Fungal diseases are referred to as mycoses. Some mycoses are endemic, i.e. infection is acquired in the geographic area that is the natural habitat of that fungus. These endemic mycoses are usually self-limited and minimally symptomatic. Some mycoses are chiefly opportunistic, occurring in immunocompromised patients such as organ transplant patients, cancer patients undergoing chemotherapy, burn patients, AIDS patients, or patients with diabetic ketoacidosis.

Fungal infections are becoming a major health concern for a number of reasons, including the limited number of antifungal agents available, the increasing incidence of species resistant to older antifungal agents, and the growing population of immunocompromised patients at risk for opportunistic fungal infections. Neutropenic patients (due to, e.g., chemotherapy, immunosuppressive therapy, infection, including AIDS, or an otherwise dysfunctional immune system) are predisposed to the development of invasive fungal infections, most commonly including Candida species and Aspergillus species, and, on occasion, Fusarium, Trichosporon and Dreschlera. Ctyptoccocus infection is also common in immunocompromised patients.

The majority of known antifungal agents fall into one of three main groups. The major group includes polyene derivatives, including amphotericin B and the structurally related compounds nystatin and pimaricin, which are only administered intravenously. These are broad-spectrum antifungals that bind to ergosterol, a component of fungal cell membranes, and thereby disrupt the membranes, leading to cell death. Amphotericin B is usually effective for systemic mycoses, but its administration is limited by toxic effects that include fever and kidney damage, and other accompanying side effects such as anemia, low blood pressure, headache, nausea, vomiting and phlebitis. The unrelated antifungal agent flucytosine (5-fluorocytosine), an orally absorbed drug, is frequently used as an adjunct to amphotericin B treatment for some forms of candidiasis and cryptococcal meningitis. Its adverse effects include bone marrow depression with leukopenia and thrombocytopenia.

The second major group of antifungal agents includes azole derivatives which impair synthesis of ergosterol and lead to accumulation of metabolites that disrupt the function of fungal membrane-bound enzyme systems (e.g., cytochrome P450) and inhibit fungal growth. Significant inhibition of mammalian P450 results in important drug interactions. This group of agents includes ketoconazole, clotrimazole, miconazole, econazole, butoconazole, oxiconazole, sulconazole, terconazole, fluconazole and itraconazole. These agents may be administered to treat systemic mycoses. Ketoconazole, an orally administered imidazole, is used to treat nonmeningeal blastomycosis, histoplasmosis, coccidioidomycosis and paracoccidioidomycosis in non-immunocompromised patients, and is also useful for oral and esophageal candidiasis. Adverse effects include rare drug-induced hepatitis; ketoconazole is also contraindicated in pregnancy. Itraconazole appears to have fewer side effects than ketoconazole and is used for most of the same indications. Fluconazole also has fewer side effects than ketoconazole and is used for oral and esophageal candidiasis and cryptococcal meningitis. Miconazole is a parenteral imidazole with efficacy in coccidioidomycosis and several other mycoses, but has side effects including hyperlipidemia and hyponatremia.

The third major group of antifungal agents includes allylamines-thiocarbamates, which are generally used to treat skin infections. This group includes tolnaftate and naftifine.

Additional groups of antifungals inhibit DNA synthesis, inhibit glucan synthesis (and hence cell wall disruption; e.g., caspofungin), and inhibit mitosis.

Another antifungal agent is griseoflulvin, a fungistatic agent which is administered orally for fungal infections of skin, hair or nails that do not respond to topical treatment.

Compounds of this invention can be employed in combination with any one or more of non-amiodarone antifungal agents known in the art and particularly with any one or more of the non-amiodarone antifungal agents described herein. More specifically, one or more of the compounds of this invention can be employed with any one or more of the antifungals of the first, second or third major groups listed above. Further, compounds of this invention can be employed in combination with any one or more of the antifungals that inhibit DNA synthesis, inhibit glucan synthesis or inhibit mitosis. Compounds of this invention can be used in combination with any one or more of griseoflulvin, echinocardins, particularly capsofungin, tolnaflate, naftifine, ketoconazole, clotrimazole, miconazole, econazole, butoconazole, oxiconazole, sulconazole, terconazole, fluconazole, itraconazole, amphotericin B and/or flucytosine (5-fluorocytosine).

Most endemic mycoses are acquired by the respiratory route and are minimally symptomatic; cough, fever, headache, and pleuritic pain may be seen. Occasionally, endemic mycoses may cause progressive pulmonary disease or systemic infection. Histoplasmosis, caused by Histoplasma, is the most common endemic respiratory mycosis in the United States; over 40 million people have been infected. The disease is noncontagious and ordinarily self-limited, but chronic pulmonary infection and disseminated infection may occur. Pulmonary infection rarely requires treatment, but disseminated infection may be treated with amphotericin B. Coccidioidomycosis, caused by Coccidioides, is a noncontagious respiratory mycosis prevalent in the southwest United States. It also is usually self-limited but may lead to chronic pulmonary infection or disseminated infection. Amphotericin B or miconazole may be given for treatment. Blastomycosis, caused by Blastomyces is a noncontagious, subacute or chronic endemic mycosis most commonly seen in the southeast United States. Most pulmonary infections are probably self-limited. Patients with progressive lung disease or disseminated disease, and immunocompromised patients, may be treated systemically with amphotericin B. Paracoccidioidomycosis, caused by Paracoccidioides, is a noncontagious respiratory mycosis that is the most common systemic mycosis in South America. It may be acute and self-limited or may produce progressive pulmonary disease or extrapulmonary dissemination. Disseminated disease is generally fatal in the absence of therapy. Sulfonamides may be used but have a low success rate. Amphotericin B produces a higher response rate but relapses may still occur.

Cryptococcosis is a noncontagious, often opportunistic mycosis. It is characterized by respiratory involvement or hematogenous dissemination, often with meningitis. A major etiologic agent is C. neoformans. Most pulmonary infections are probably overlooked, but cryptococcal meningitis, which accounts for 90% of reported disease, is dramatic and seldom overlooked. Cryptococcosis is a particular problem in immunocompromised patients; cryptococcal meningitis occurs in 7 to 10% of AIDS patients. The principal symptom of meningitis is headache; associated findings include mental changes, ocular symptoms, hearing deficits, nausea, vomiting, and seizures. Without treatment, 80% of patients die within two years. In meningitis, cryptococci can be observed in India ink preparations of cerebrospinal fluid sediment, and can be cultured from the cerebrospinal fluid. Treatment is generally with fluconazole or the combination of amphotericin B and flucytosine, although amphotericin B does not cross the blood brain barrier.

Aspergillosis is a term that encompasses a variety of disease processes caused by Aspergillus species. Aspergillus species are ubiquitous; their spores are constantly being inhaled. Of the more than 300 species known, only a few are ordinarily pathogenic for man: A. fumigatus, A. flavus, A. niger, A. nidulans, A. terreus, A. sydowi, A. flavatus, and A. glaucus. Aspergillosis is increasing in prevalence and is particularly a problem among patients with chronic respiratory disease or immunocompromised patients. Among immunocompromised patients, aspergillosis is second only to candidiasis as the most common opportunistic mycosis and accounts for about 15% of the systemic mycoses in this group. Opportunistic pulmonary aspergillosis is characterized by widespread bronchial erosion and ulceration, followed by invasion of the pulmonary vessels, with thrombosis, embolization and infarction. Clinically, infection manifests as a necrotizing patchy bronchopneumonia, sometimes with hemorrhagic pulmonary infarction. In about 40% of cases, there is hematogenous spread to other sites. Aspergillosis is also a rare but devastating complication of burn wounds; amputation is often required for cure. Invasive aspergillosis is commonly fatal, so aggressive diagnosis and treatment is required. Blood, urine and cerebrospinal fluid cultures are rarely positive, but fungi can be seen in smears and biopsies. Amphotericin B can be given for treatment.

Dermatophytosis is a chronic fungal infection of the skin, hair or nails by dermatophytes, which include members of the species Trichophyton, Microsporum and Epidermophyton. Infection of the foot (tinea pedis), scalp (tinea capitis) are most common, although widespread infection on non-hair-bearing skin (tinea corporis) also occurs. Clinical manifestations vary and may present on the skin as fissuring or lesions with scaling, vesicles or pustules (and alopecia on the scalp), or on the nails as discolored or chalky, crumbling nails. Both topical and systemic therapies may be used to treat dermatophyte infection, including topically administered imidazoles and triazoles (such as itraconazole, miconazole, ketoconzaole and econzaole), haloprogin, undecylic acid, ciclopirox olamine, tolnaftate and terbinafine.

Fusarium species can cause localized or hematogenously disseminated infection (fusariosis), most frequently in patients who have a hemopoietic malignancy and neutropenia. Abrupt onset of fever, sometimes with myalgia, is followed in the majority of cases by distinctive skin lesions resembling ecthyrna gangrenosum. Infection can be treated with amphotericin B but recovery depends ultimately on alleviation of neutropenia. Mortality typically exceeds 90%.

Mucormycosis is an acute suppurative opportunistic mycosis that produces rhinocerebral, pulmonary or disseminated disease in immuno-compromised patients, and local or disseminated disease in patients with burns or open wounds. Infection is caused by fungi in the class Zygomycetes, and include Basidiobolus, Conidiobolus, Rhizopus, Mucor, Absidia, Mortierella, Cunninghamella, and Saksenaea. Rhinocerebral mucormycosis accounts for about half of all cases of mucormycosis. It is one of the most rapidly fatal fungal diseases, with death occurring within 2-10 days in untreated patients. Early clinical signs include nasal stuffiness, bloody nasal discharge, facial swelling and facial pain. The infection then spreads to the eyes, cranial nerves and brain. Pulmonary mucormycosis is nearly as common as rhinocerebral disease and manifests with the same necrotizing and infarction as aspergillosis. Fungi are virtually never seen or cultured from blood, sputum or cerebrospinal fluid. Disseminated mucormycosis may follow pulmonary or burn wound infection. Treatment is with amphotericin B.

Candidiasis is a general term for a variety of local and systemic processes caused by colonization or infection of the host by species of the yeast Candida. Candidiasis occurs worldwide; superficial infections of the skin, mouth and other mucus membranes are universal. Invasive systemic disease has become a problem due to the use of high doses of antibiotics that destroy normal bacterial flora, immunosuppressive agents, and agents toxic to bone marrow, e.g., during cancer therapy. Neutropenia is a major risk factor for Candida dissemination. Candidiasis is also seen among immunocompromised individuals such as AIDS patients, organ transplant patients, patients receiving parenteral nutrition, and cancer patients undergoing radiation treatment and chemotherapy. It is the most common opportunistic mycosis in the world. The most common etiologic agent is Candida albicans. Other infectious species include C. tropicalis, C. parapsilosis, C. stellatoidea, C. kusei, C. parakwsei, C. lusitaniae, C. pseudotropicalis, C. guilliermondi, C. guilliermondii, C. viswanathii, and C. glabrata. Candida albicans is normally found in the mouth, throat, gastrointestinal tract and vagina of humans. Candida glabrata is the second most common Candida pathogen after C. albicans, causing infections of the urogenital tract, and of the bloodstream (Candidemia). Non-albicans species frequently colonize skin. Candida species occur in two forms that are not temperature- or host-dependent. The usual colonizing forms are yeasts that may assume a pseudomycelial configuration, especially during tissue invasion. Pseudomyceliae result from the sequential budding of yeasts into branching chains of elongated organisms.

Candida albicans contains cell wall mannoproteins that appear to be responsible for attachment of the yeast cells to specific host tissues. It has been reported that the mannosyl residues, rather than the protein portion, of the mannoproteins is responsible for adherence of fungal cells to spleen and lymph node tissues in mice. (Kanbe, et al., 1993,).

C. albicans also binds avidly to extracellular matrix (ECM) proteins such as fibronectin, laminin, and types I and IV collagen, all of which contain heparin-binding domains. This suggests C. albicans may express a heparin-like surface molecule. Adherence of C. albicans to the ECM may be important in the pathogenesis of disseminated candidiasis. It has been demonstrated that heparin, heparan sulfate and dextran sulfate glycosaminoglycans (GAGs) inhibit adherence of C. albicans to ECM and ECM proteins, possibly by a mechanism involving binding of GAGs to ECM proteins, thus masking these selective ligands. (Klotz and Smith, 1992).

Clinically, candidiasis manifests as superficial mucocutaneous infections, chronic mucocutaneous candidiasis, or systemic infection. Superficial mucocutaneous infections can occur in any area of skin or mucus membrane. Thrush, commonly seen in AIDS patients, is characterized by a patchy or continuous, creamy to gray pseudomembrane that covers the tongue, mouth, or other oropharyngeal surfaces and may be accompanied by ulceration and necrosis. Laryngeal involvement results in hoarseness. Esophagitis is often an extension of oropharyngeal disease and may manifest with symptoms of retrosternal pain and dysphagia. Intestinal candidiasis is commonly asymptomatic, but is a major source of hematogenous invasion in immunocompromised individuals. Intertrigo involves the axillae, groins, inframammary folds, and other warm, moist areas, and may manifest as red, oozing or dry, scaly lesions. Infections may occur in other areas, including perianal and genital areas. Paronychia, infection of the nails, often follows chronic exposure of the hands or feet to moisture. Some patients with limited T-cell immunodeficiency develop chronic mucocutaneous candidiasis. These patients suffer from persistent superficial Candida infection of the skin, scalp, nails and mucus membranes.

Most cases of systemic candidiasis are caused by Candida albicans and C. tropicalis, and increasingly, C. glabrata. Clinical manifestations of Candida infection appear mainly in the eyes, kidneys and skin. In the eyes, there may be single or multiple raised, white, fluffy chorioretinal lesions. These lesions are a potential cause of blindness. Involvement of the kidneys includes diffuse abscesses, capillary necrosis and obstruction of the ureters. Infection may result in progressive renal insufficiency. Systemic Candida infection can also manifest as maculonodular skin lesions surrounded by a reddened area; these lesions have an appearance similar to acne but are a major clue to a potentially lethal disease. Other manifestations of systemic candidiasis may include osteomyelitis, arthritis, meningitis, and abscesses in the brain, heart, liver, spleen and thyroid. Involvement of the lungs is also common, but pulmonary lesions are usually too small to be seen on chest X-ray. Finally, Candida endocarditis can occur in patients receiving prolonged intravenous therapy or cardiac valve implants, or in intravenous drug abusers. Fungal lesions appear on the valves, and can embolize and occlude large blood vessels.

Superficial infections are diagnosed by microscopic examination of scrapings or swabs of infected lesions in the presence of 10% potassium hydroxide. Candida organisms can also be seen on gram stain. Endocarditis is diagnosed by blood cultures or demonstration of bulky valvular lesions on echocardiography. Systemic candidiasis may be difficult to diagnose because the presence of heavy colonization at the usual sites of infection indicates, but does not prove, that dissemination has occurred. The most reliable evidence of systemic candidiasis is biopsy demonstration of tissue invasion or recovery of yeast from fluid in a closed body cavity, such as cerebral spinal fluid, pleural or peritoneal fluid. Similarly, positive blood or urine or sputum cultures may indicate invasive disease or simply localized disease around indwelling devices, e.g., catheters or intravenous lines.

Mucocutaneous infections may be treated with topical preparations of nystatin, amphotericin B, clotrimazole, miconazole, haloprogin or gentian violet. Oropharyngeal or esophageal candidiasis can be treated with systemic agents such as ketoconazole or fluconazole. Chronic mucocutaneous candidiasis syndrome may respond to topical or systemic therapeutic agents such as amphotericin B or ketoconazole, but often relapses when medication is discontinued. Cystitis may be treated with amphotericin B bladder rinses, or a brief low-dose intravenous course of amphotericin B with or without oral flucytosine. Endocarditis is essentially incurable without valve replacement, accompanied by a 6 to 10 week course of amphotericin B and flucytosine. Even with therapy, however, complete cure of endocarditis is not always possible.

The mortality rate from systemic candidiasis is about 50% despite the availability of new antifungal drugs. Systemic candidiasis may be treated with fluconazole, a fungistatic agent, or amphotericin B, a fungicidal agent although systemic use of the latter is limited by its toxicity. Both drugs have substantial adverse reactions when used in combination with cyclosporine A, which itself can be nephrotoxic. The removal of precipitating factors such as intravenous lines or catheters is also important for controlling infection. Flucytosine therapy can be added to the amphotericin B therapy for treatment of systemic candidiasis, especially in patients that are not immunocompromised. In immunocompromised patients, however, these infections are problematic and resist effective treatment. Mortality with systemic candidiasis can be over 90% in such patients. Furthermore, chronic mucocutaneous candidiasis and candidal endocarditis often show evidence of disease after having been declared cured.

Infection of the cornea and conjunctiva, including keratoconjunctivitis, can result from infection by amoeba, viruses, fungi and bacteria. Debilitated patients can develop keratitis from fungi such as Candida or Fusarium which is often associated with corneal ulceration and can lead to scarring with severe visual loss.

Amiodarone is a Class III antiarrhythmic drug (BeDell, 1996; USPDI, 1997). It is administered orally in 200 mg tablets and is sold under the name Cordarone (Trademark, Sanofi-Aventis Corp.) in the United States and several other brand names outside the U.S. Amiodarone prolongs the repolarization phase of the cardiac action potential. Amiodarone has beneficial effects in the treatment of patients with ventricular arrhythmias after myocardial infarction. Amiodarone therapy for arrhythmia requires prolonged treatment on the order of months to years. Amiodarone dosed for this use has several unwanted side-effects, including thyroid dysfunction, hepatitis, impaired vision, photosensitivity of the skin, and pneumonitis.

In animals, amiodarone is effective in the prevention or suppression of experimentally induced arrhythmias. The antiarrhythmic effect of amiodarone may be due to at least two major properties: 1) a prolongation of the myocardial cell-action potential duration and refractory period; and 2) noncompetitive alpha- and beta-adrenergic inhibition. Amiodarone prolongs the duration of the action potential of all cardiac fibers while causing minimal reduction of dV/dt. The refractory period is prolonged in all cardiac tissues.

Following oral administration in man, amiodarone is slowly and variably absorbed. The bioavailability of amiodarone is approximately 50% but has varied between 35 and 65% in various studies. Maximum plasma concentrations are attained 3 to 7 hours after a single dose. Despite this, the onset of amiodarone's antiarrhythmic effect may take several days to 1 to 3 weeks to occur, even when administered with a loading dose. Plasma concentrations with chronic dosing at 100 to 600 mg/day are approximately dose proportional, with a mean 0.5 mg/L increase for each 100 mg/day administered. However, considerable individual variability occurs.

Amiodarone has a very large, but variable volume of distribution, averaging about 60 L/kg because of extensive accumulation in various sites, especially adipose tissue and highly perfused organs, such as the liver, lung, and spleen. One major metabolite of amiodarone, desethylamiodarone, has been identified in man; it accumulates to an even greater extent in almost all tissues. The pharmacological activity of this metabolite, however, is not known. During chronic treatment, the plasma ratio of metabolite to parent compound is approximately one. The main route of amiodarone elimination is via hepatic excretion into bile.

There may be significant adverse side effects with the use of amiodarone. These include neurotoxicity, photosensitivity, and pulmonary fibrosis or interstitial pneumonitis/alveolitis. Ataxia is the most common symptom, occurring in 20-40% of patients, especially during administration of loading doses. It may occur within 1 week to several months after initiation of therapy and may persist for more than a year after withdrawal. Photosensitivity may require ultraviolet-A sun-block, such as zinc or titanium oxide and protective clothing. Pulmonary fibrosis or interstitial pneumonitis/alveolitis is clinically significant in up to 10 to 15% of patients, but abnormal diffusion capacity occurs in a much higher percentage of patients, more frequently with doses of 400 mg/day and after several months of treatment. It is usually reversible after withdrawal of amiodarone but is fatal in about 10% of cases, especially when not diagnosed promptly.

Incidence of adverse side effects is generally related to dose and duration of therapy. Adverse side effects may occur even at therapeutic plasma amiodarone concentrations, but are more common at concentrations over 2.5 mg/l and with continuous treatment for longer than 6 months.

Amiodarone has also been found to affect mammalian G protein activity by (Hageluken, et al., 1995, Mol. Pharmacol. 47: 234-240) who reported that amiodarone increased high affinity GTP hydrolysis with an EC50 of 7.5 μM and stimulated binding of guanosine-5μ-O-(3-thio)triphosphate to, and incorporation of GTP azidoanilide into Gi protein subunits in HL-60 membranes. These authors also found that amiodarone increased the cytosolic Ca²⁺ concentration in HL-60 cells in the presence but not in the absence of extracellular Ca²⁺. In vitro, amiodarone activated the GTPase of reconstituted Gi/Go proteins and Gi2 with EC50 values of 20 μM and 50 μM, respectively. These authors concluded that amiodarone is a direct activator of Gi and Go proteins and that amiodarone activates nonselective cation channels in HL-60 cells via Gi proteins.

Pharmaceutical compositions of this invention and useful in methods herein comprise an one or more compounds of this invention with antifungal properties and a pharmaceutically acceptable excipient, diluent, adjuvant or carrier and are administered topically, intrperitoneally, intravenously, orally or as an aerosol. The antifungal compounds are present in the composition in a therapeutically effective amount or a combined therapeutically effective amount. Additional pharmaceutical compositions comprise in addition to the one or more compounds of Formula I a known antifungal agent as described herein. More specifically that known antifungal agent is an amiodarone compound and yet more specifically is amiodarone or a salt thereof.

In vitro methods of the invention permit killing or inhibiting replication of fungi through contacting the fungi with one or more compounds of this invention or with a pharmaceutical composition containing the same. Fungal infection treatment methods of the invention comprise administering to a subject suffering from a fungal infection a therapeutically effective amount of a compound of this invention and such treatment methods are applicable to fungal infection, including, for example, infection by Cryptococcus, Aspergillus or Candida (especially, C. albicans, C. glabrata, C. krusei, C. lusitaniae, C. parapsilosis and C. tropicalis) species and others as described herein. Therapeutically effective amounts include amounts effective to inhibit replication of or kill fungi.

Administration of compounds of this invention may be especially beneficial in immunocompromised patients, including immuno suppressed and neutropenic patients, for example, patients undergoing chemotherapy, radiation therapy, or immunosupressive therapy, or patients with a dysfunctional immune system secondary to infection, such as HIV infection, or other causes. Topical administration of compounds of the invention is also expected to be effective for treating a variety of fungal infections, including, for example, skin and eye infections, including those caused by dermatophytes.

Medicaments/pharmaceutical compositions useful according to the invention can include other antifungal agents including non-amiodarone agents or can be used in combinative therapeutic methods with other such agents.

An antifungal activity of a compound of this invention in in vitro and/or in vivo methods/medicaments of the invention includes antifungal activity, for example, against one or more species of Candida, Aspergillus, Cryptococcus, Histoplasma, Coccidioides, Blastomyces, Basidiobolus, Conidiobolus, Rhizopus, Rhizomucor, Mucor, Absidia, Mortierella, Cunninghamella, Saksenaea, Fusarium, Trichophyton, Trichosporon, Microsporum, Epidermophyton, Scytalidium, Malassezia, Actinomyceees, Sporothrix and Penicillium.

Antifungal activity with respect to plant pathogens includes antifungal activity (including fungistatic and fungicidal activity), for example, against one or more species of Pythium, Phytophthora, Fusarium, Rhizoctona, Thielaviopsis, Cylindrocladium, Gibberella, Sclerotinia, Collectotrichium or Aspergillus.

A compound according to the invention may be administered systemically or topically. Systemic routes of administration include oral, intravenous, intramuscular or subcutaneous injection (including into depots for long-term release), intraocular or retrobulbar, intrathecal, intraperitoneal (e.g. by intraperitoneal lavage), transpulmonary using aerosolized or nebulized drug, or transdermal. Topical routes include administration in the form of ointments, gels, salves, ophthalmic drops, ear drops, or irrigation fluids (for example, irrigation of wounds).

Those skilled in the art can readily optimize effective dosages and administration regimens for compositions comprising a compound of this invention as determined by good medical practice and the clinical condition of the individual subject.

In a specific embodiment, compounds of this invention are useful in treatment of pulmonary aspergillosis, particularly when administered in combination with an amiodorone compound. Compounds of this invention are particularly useful in treatment of pulmonary aspergillosis that occurs in patients that have under gone bone marrow transplantation (BMT).

Antifungal compounds and compositions useful according to the invention may be administered in conjunction with other antifungal agents. These non-amiodarone antifungal agents may include azole derivatives such as the imidazoles and triazoles (e.g., ketoconazole, clotrimazole, miconazole, econazole, butoconazole, oxiconazole, sulconazole, terconazole, fluconazole and itraconazole); amphotericin B, nystatin and pimaricin; flucytosine (5-fluorocytosine); allylamines-thiocarbamates, such tolnaftate and naftifine; griseofulvin, haloprogin, undecylic acid, ciclopirox olamine, tolnaftate and terbinafine.

Concurrent administration of a compound of this invention of Formula I with other (e.g., amiodarone and non-amiodarone) antifungal agents is expected to improve the therapeutic effectiveness of the antifungal agents. This may occur through reducing the concentration of antifungal agent required to eradicate or inhibit fungal growth, e.g., replication. Because the use of some agents is limited by their systemic toxicity or prohibitive cost, lowering the concentration of antifungal agent required for therapeutic effectiveness reduces toxicity and/or cost of treatment, and thus allows wider use of the agent. Concurrent administration of a compound of this invention of Formula I with another antifungal agent may produce a more rapid or complete antifungal/fungicidal effect than could be achieved with either agent alone. Administration of a compound of Formula I may reverse the resistance of fungi to antifungal agents. Administration of a compound of Formula I may also convert a fungistatic agent into a fungicidal agent.

An advantage provided by the present invention is the ability to treat fungal infections, particularly fungal infections from Cryptococcus, Aspergillus, or Candida, which are presently considered incurable. Another advantage is the ability to treat fungi that have acquired resistance to known antifungal agents. A further advantage of concurrent administration of a compound of this invention with an antifungal agent having undesirable side effects, e.g., amphotericin B, is the ability to reduce the amount of antifungal agent needed for effective therapy. The present invention may also provide quality of life benefits due to, e.g., decreased duration of therapy, reduced stay in intensive care units or reduced stay overall in the hospital, with the concomitant reduced risk of serious nosocomial (hospital-acquired) infections.

Concurrent administration, as used herein, includes administration of the agents together, simultaneously or before or after each other. A compound of Formula I herein and other antifungal agents may be administered by different routes. For example, the compound of Formula I may be administered intravenously while the other antifungal agents are administered intramuscularly, intravenously, subcutaneously, orally or intraperitoneally. Alternatively, the compound of Formula I may be administered intraperitoneally while the other antifungal agents are administered intraperitoneally or intravenously, or the amiodarone compound may be administered in an aerosolized or nebulized form while the other antifungal agents are administered, e.g., intravenously. All of the antifungal agents may be both administered orally. The compound of Formula I and antifungal agents may be given sequentially in the same intravenous line, after an intermediate flush, or may be given in different intravenous lines. The compound of Formula I and antifungal agents may be administered simultaneously or sequentially, as long as they are given in a manner sufficient to allow both agents to achieve effective concentrations at the site of infection.

Concurrent administration of the compound of Formula I and another antifungal agent is expected to provide more effective treatment of fungal infections. Concurrent administration of the two agents may provide greater therapeutic effects in vivo than either agent provides when administered singly. For example, concurrent administration may permit a reduction in the dosage of one or both agents with achievement of a similar therapeutic effect. Alternatively, the concurrent administration may produce a more rapid or complete fungicidal/fungistatic effect than could be achieved with either agent alone.

Therapeutic effectiveness is based on a successful clinical outcome, and does not require that the antifungal agent or agents kill 100% of the organisms involved in the infection. Success depends on achieving a level of antifungal activity at the site of infection that is sufficient to inhibit the fungi in a manner that tips the balance in favor of the host. When host defenses are maximally effective, the antifungal effect required may be minimal. Reducing organism load by even one log(a factor of 10) may permit the host's own defenses to control the infection. In addition, augmenting an early fungicidal/fungistatic effect can be more important than a long-term fungicidal/fungistatic effect. These early events are a significant and critical part of therapeutic success, because they allow time for host defense mechanisms to activate.

The invention provides a method of killing or inhibiting growth of fungi comprising contacting the fungi with a compound of Formula I according to the invention. This method can be practiced in vivo or in a variety of in vitro uses such as in agricultural uses or food preparations or to decontaminate fluids and surfaces or to sterilize surgical and other medical equipment and implantable devices, including prosthetic joints. These methods can also be used for in situ sterilization of indwelling invasive devices such as intravenous lines and catheters, which are often foci of infection.

A further aspect of the invention involves use of a compound of Formula I for the manufacture of a medicament for treatment of fungal infection. The medicament may include, in addition to such compounds of Formula I, an amiodarone compound or other chemotherapeutic agents such as non-amiodarone antifungal agents. The medicament can optionally comprise a pharmaceutically acceptable excipient, diluent, adjuvant or carrier. Medicaments of this invention are useful for human or veterinary treatment.

The administration of antifungal compounds of this invention is suitably accomplished with a pharmaceutical composition comprising one or more compounds of Formula I and a pharmaceutically acceptable excipient, diluent, adjuvant, or carrier. The antifungal compounds or combinations of antifungal compounds of this invention may be administered without or in conjunction with known surfactants, other chemotherapeutic agents or additional known antifungal agents.

Agriculturally useful compositions of this invention comprise one or more compounds of this invention which have antifungal properties in an amount or combined amount effective for preventing fungal infestation or for killing or limiting the growth of one or more fungal species, and an agriculturally acceptable excipient, diluent, adjuvant or carrier. In particular, agricultural formulations include solutions and suspensions, as well as emulsions containing the active antifungal agent. In specific embodiments, compounds of this invention can be administered in solution in DMSO or alcohols, e.g., ethanol. Such compositions can be administered by spraying (such as in a solution suspension or as an aerosol), or dusting (as with a dry formulation).

The antifungal compounds are present in the antifungal compositions of this invention in an amount or a combined amount that is effective to prevent or limit growth of one or more fungal species in the application (in vivo, in plants, etc.) contemplated. The compositions may be fungistatic or fungicidal.

Additional agricultural compositions comprise in addition to the one or more compounds of Formula I an art known plant antifungal agent.

In reference to the descriptions of variables in chemical formula herein:

The term “alkyl” refers to a monoradical of a branched or unbranched (straight-chain or linear) saturated hydrocarbon and to cycloalkyl groups having one or more rings. Unless otherwise indicated preferred alkyl groups have 1 to 20 carbon atoms and more preferred are those that contain 1-12 carbon atoms. Short alkyl groups are those having 1 to 6 carbon atoms including methyl, ethyl, propyl, butyl, pentyl and hexyl groups, including all isomers thereof. Long alkyl groups are those having 8-20 carbon atoms and preferably those having 12-20 carbon atoms as well as those having 12-20 and those having 16-18 carbon atoms. The term “cycloalkyl” refers to cyclic alkyl groups having preferably 3 to 20 carbon atoms having a single cyclic ring or multiple condensed rings. Cycloalkyl groups include, by way of example, single ring structures such as cyclopropyl, cyclobutyl, cyclopentyl, cyclohexyl, cyclooctyl, and the like, or multiple ring structures such as adamantanyl, and the like. Unless otherwise indicated alkyl groups including cycloalkyl groups are optionally substituted as defined below.

The term “aryl” refers to a monoradical containing at least one aromatic ring. The radical is formally derived by removing a H from a ring carbon. Aryl groups contain one or more rings at least one of which is aromatic. Rings of aryl groups may be linked by a single bond or a linker group or may be fused. Exemplary aryl groups include phenyl, biphenyl and naphthyl groups. Aryl groups include those having from 6 to 20 carbon atoms and those containing 6-12 carbon atoms. Unless otherwise noted aryl groups are optionally substituted as described herein.

The term “sulfonate” refers to the radical —SO₃—R″ where R″ is hydrogen, alkyl, alkenyl, alkynyl, aryl, heterocyclyl, or heteroaryl radical as described above. An “alkyl sulfonate” group refers to a sulfonate group wherein R″ is alkyl. An “aryl sulfonate” group refers to a sulfonate group wherein R″ is aryl. Alkyl, alkenyl, alkynyl, aryl, heterocyclyl, or heteroaryl groups can be optionally substituted as described below. The group —SO₃H can be in the ionic form —SO₃⁻, or in the form of salt with a counterion.

The term “amino” refers to the group —NH₂ as well as to —NRR′ groups where R and R′ are hydrogen, or optionally substituted alkyl, alkenyl, alkynyl or aryl groups. Preferred R and R′ are hydrogen, alkyl (particularly alkyl having 1-6 or 1-3 carbons), and aryl groups (particularly those which having 1 or two 5- or 6-member rings, particularly those which are phenyls, naphthalenes, and particularly those which are not heteroaryl groups). Amino groups may be protonated or quaternized to form positively charged species which can be isolated in the form of salts with appropriate counter ions (halide ions (e.g., Cl⁻) carboxylate ions (e.g., RCOO⁻, where R is optionally substituted alkyl and aryl, e.g. trifluoroacetate) as is known in the art

Haloalkyl” refers to alkyl as defined herein substituted by one or more halides (e.g., F—, Cl—, I—, Br—) as defined herein, which may be the same or different. A haloalkyl group may, for example, contain 1-10 halide substituents. Representative haloalkyl groups include, by way of example, trifluoromethyl, 3-fluorododecyl, 12,12,12-trifluorododecyl, 2-bromooctyl, 3-bromo-6-chloroheptyl, and the like. Haloalkyl groups include bromoalkyl groups and trifluoromethyl groups among others.

Alkyl, alkenyl, alkynyl, aryl, heterocyclic and heteroaryl groups may be substituted or unsubstituted. Alkyl and aryl groups may be optionally substituted as described herein and may contain non-hydrogen substituents dependent upon the number of carbon atoms in the group and the degree of unsaturation of the group. Unless otherwise indicated substituted groups preferably contain 1-10, 1-6 or 1, 2 or 3 non-hydrogen substituents. Unless otherwise indicated substituted alkyl and aryl groups preferably contain 1-10, and more preferably 1-6, and more preferably 1, 2 or 3 non-hydrogen substituents.

Optional substitution refers to substitution with one or more of the following functional groups: alkyl (particularly alkyl having 1-6 carbon atoms); hydroxyl groups, halogens, amino groups, aryl (particularly phenyl or benzyl) alkyl sulfonate, and aryl sulfonate groups.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

The compounds of this invention may contain one or more chiral centers. Accordingly, this invention is intended to include racemic mixtures, diasteromers, enantiomers and mixture enriched in one or more stereoisomer. The scope of the invention as described and claimed encompasses the racemic forms of the compounds as well as the individual enantiomers and non-racemic mixtures thereof.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. A number of specific groups of variable definitions have been described herein. It is intended that all combinations and subcombinations of the specific groups of variable definitions are individually included in this disclosure.

When a compound is described herein such that a particular isomer, enantiomer or diastereomer of the compound is not specified, for example, in a formula or in a chemical name, that description is intended to include each isomers and enantiomer of the compound described individual or in any combination.

Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants, including those carrying radioisotopes, may also be useful in diagnostic assays and in therapeutics. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound of the formula I with an amount of acid or base, such as an equivalent amount, in a medium such as one in which the salt precipitates or in an aqueous medium followed by lyophilization. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Every formulation or combination of components described or exemplified herein can be used to practice the invention, unless otherwise stated. Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. Each reference cited herein is incorporated by reference herein in its entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention.

### THE EXAMPLES

### Example 1

### Materials and Methods

### Strains, Media, and Reagents

The Cryptococcus neoformans strain used was JEC21 (MATα). The Aspergillus fumigatus strain used was AF293. Cells were grown in SD (0.17% w/v Difco yeast nitrogen base without amino acids and ammonium sulfate, 0.4% w/v ammonium sulfate, 2% w/v glucose). Agar media contained 2% w/v Bacto-agar. Adenine (12 mg/L final concentration), uridine (40 mg/L), leucine (30 mg/L), histidine (20 mg/L), and tryptophan (20 mg/L) were added to supplement auxotrophies as needed.

### Growth Rates and Viability.

For quantitation of Cryptococcus cell proliferation, cells were grown in 5 ml of liquid medium in Klett test tubes with vigorous shaking in a water bath. Measurement of cell density was done in a Klett-Summerson colorimeter. Cells were grown overnight in the medium to be tested. Dilutions of the overnight cultures were made into a series of test tubes containing fresh medium and various concentrations of drug or DMSO. The cell densities were monitored at time zero (typically about 1-2×10⁶ cells/ml) and over time (typically 8-10 points for each growth curve) up to about 10 hours. The increase in cell density was plotted versus time and the resulting curves were used to determine the generation time for each culture (Stanier et al., 1976). Each experiment was repeated 3 or more times and the mean values for generation times are given in the text and tables along with their standard errors.

Growth of Aspergillus was measured by inoculating 10⁵ spores into liquid minimal growth medium (5-20 ml) containing amiodarone, AR3, amiodarone plus AR3, or only the carrier DMSO and incubated at 30° C. with vigorous shaking in a water bath. After 4 days of growth, the cultures were removed from the liquid medium, dried and total culture weight determined. The mean values of four experiments were determined.

### Aequorin Luminescence.

Cells were transformed with pEVP11/AEQ, which constitutively expresses apoaequorin directed to the cytoplasm (Batiza et al., 1996) Transformed cells were grown to mid-exponential phase in SD-leu to maintain the plasmid. 1.5×10⁷ cells were pelleted and resuspended in fresh SD-leu medium containing 5.9 μM coelenterazine for 1 hour to allow the cofactor to diffuse into cells and bind the apoaequorin. Cells were pelleted again and resuspended in fresh SD-leu (“SD” buffer) and 2.5×10⁶ cells per well aliquoted to a microtiter plate for luminescence measurement. Luminescence was measured in an EG&G Berthold luminometer using Winglow monitoring and analysis software.

The luminescence of aliquoted cells prior to any treatment was measured as a control, then, at 30 seconds, wells were injected with a volume of buffer equal to that of the cells and luminescence monitored for an additional 3 min. The injected buffer was SDE (SD buffer+12% EtOH)+amiodarone (100 μM resulting in 50 μM final concentration).

### Example 2

### In Vitro Antifungal Activity of AR3

Various benzofuran derivatives were screened for antifungal effect by testing their ability to inhibit the growth of C. neoformans. Table 1 shows the generation times of C. neoformans strain JEC21 growing in minimal media in the presence of one of these benzofuran derivatives called AR3 (Compound B) or in the presence of only the carrier (DMSO) as a control. When AR3 was added at 20 μM, yeast cell growth arrested immediately. The viability of the growth arrested cells was tested by the Live/Dead Lumofungin assay and the analog-treated cells were found to be dead (data not shown). JEC21 cells treated with 10 μM and 5 μM AR3 had generation times 200% and 15% slower than controls. Thus, AR3 exerts a strong inhibitory effect on the growth of C. neoformans, requiring about a 2 fold higher concentration than amiodarone.

TABLE 1
Effect of AR3 on Generation Time of Cryptococcus neoformans¹

| | 20 μM | 10 μM | 5 μM |
|---|---|---|---|
| DMSO | 188.9 (12.5) | 182 910.2) | 185.7 (14.1) |
| AR3 | No growth | 394.6 (125) | 276.1 (16.8) |

¹C. neoformans strain JEC21 was grown at 30° C. in minimal medium containing the indicated concentration of AR3 (added in 10 μl, 5 μl, or 2.5 μl DMSO, respectively) or an equal volume of only DMSO as a control. The increase in culture densities were measured over 6-10 hour periods. The generation times (in minutes) were determined by linear regression with reliability values of 0.9 or above. The mean generation times of three independent experiments are shown along with the standard errors.

### Example 3

### Effects on Mammalian Cells In Vitro

Since it is desirable to identify antifungal agents having reduced toxicity toward mammalian cells the effect of AR3 on the growth of the human cell line K-562 was assessed.

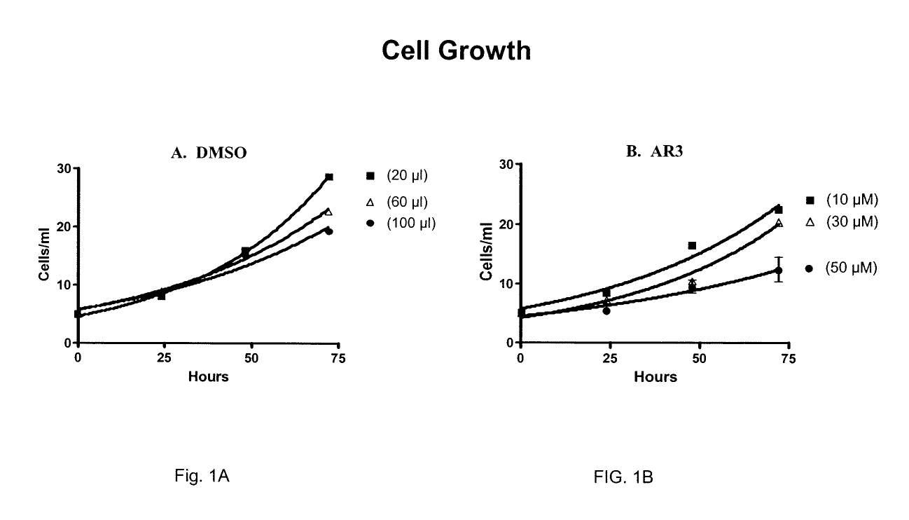

K-562 cells were grown in Iscove's modified Dulbecco's medium in culture flasks kept at 37° C. Cells were treated with AR3, amiodarone, or the carrier, DMSO, as a control. The growth of the cells was followed over a 3 day period. Control cells increased in density about 5 fold during this time (FIG. 1A). AR3 showed little toxicity to K-562 cells. Cells treated with both 10 μM and 30 μM AR3 increased more than 4 fold during the period of the experiment (FIG. 1B), similar to the growth of the control. When AR3 was added at 50 μM, cell growth was slowed slightly. AR3 treated cells had a 54.2 hours (standard error of 10.4) generation time compared to 34.0 (2.5) hours for control cells, a 60% increase.

Thus, AR3 shows no negative effects on growth of K-562 cells at concentrations (10-30 μM) that are completely inhibitory to yeast growth and shows only mild growth inhibition at a higher concentration.

### Example 4

### Effects on Yeast Cytoplasmic Ca²⁺

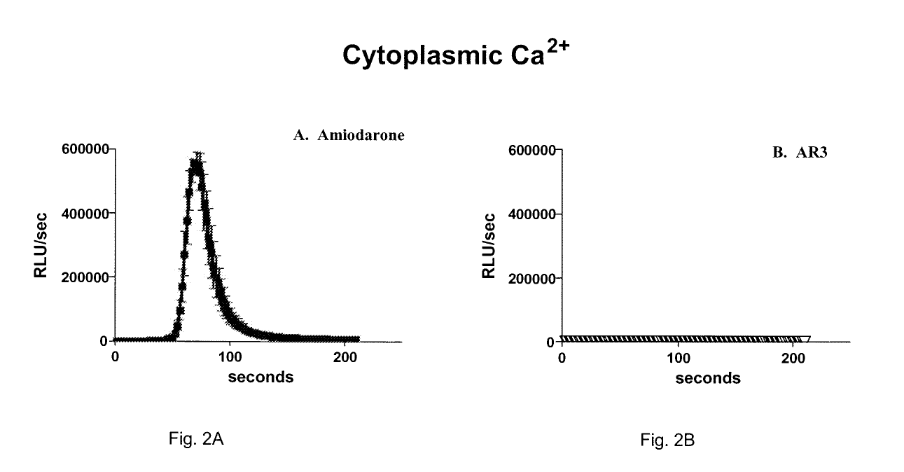

Amiodarone causes a major rise in the cytoplasmic concentration of Ca²⁺ ([Ca²⁺]cyt) in Saccharomyces cerevisiae cells (Courchesne and Ozturk, 2003). We tested for similar effects on the [Ca²⁺cyt in S. cerevisiae by treatment with AR3 (FIGS. 2A-B). S. cerevisiae cells (FY70 [pEVP11/AEQ]) expressing apoaequorin were assayed for their response to amiodarone and AR3, as described above.

Yeast cells were treated with 50 μM amiodarone or 50 μM AR3 at t=30 sec. and the increase in RLUs/sec were monitored for an additional 3.5 min. The increase in RLUs is proportional to the increase in the [Ca²⁺]cyt. Amiodarone elicited an immediate and very large increase in RLUs (FIG. 2A). In contrast, AR3 elicited no increase in the RLUs (FIG. 2B) producing a response like that of the control cells treated with carrier only.

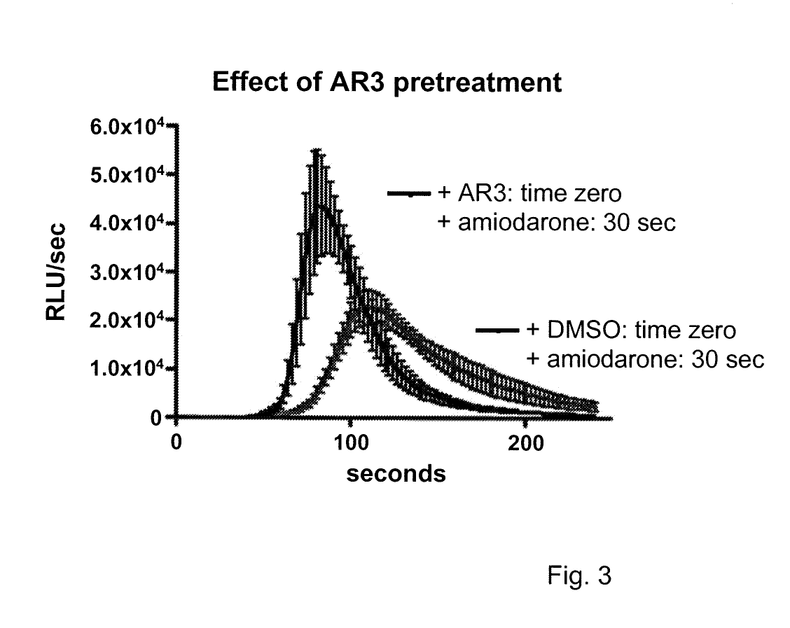

It was also investigated whether pretreatment of yeast cells with AR3 could affect the calcium response elicited by amiodarone (FIG. 3). S. cerevisiae cells were prepared for aequorin assays as just described. Control cells were pretreatment with carrier only (DMSO) at zero time and then amiodarone was added at 30 sec. This resulted in the same increase in RLUs elicit by amiodarone addition with no pretreatment. Pretreatment with AR3 increased the maximum RLUs nearly 100% and shifted significantly the response to a more rapid rise in Ca²⁺. Thus, although AR3 could not elicit a calcium mobilization by itself, it did significantly potentiate the rise in cytoplasmic calcium caused by amiodarone treatment demonstrating a synergistic effect of AR3 with amiodarone.

### Example 5

### Combination Treatment: AR3 with Amiodarone

It is clear from the results in FIG. 3 that AR3 synergizes with amiodarone for its effects on cytoplasmic calcium in S. cerevisiae. Therefore, synergism of the compounds against the pathogenic fungus Aspergillus fumigatus was tested. This fungus was chosen because amiodarone has been shown to inhibit growth of Aspergillus. Importantly, there is a need to identify new treatments for aspergillosis in many patient populations

Aspergillus cells were grown in minimal medium at 30° C. with vigorous aeration. Cultures were inoculated with 10⁵ conidia plus either 30 μl of DMSO only, 15 μM amiodarone (in DMSO), 25 μM AR3 (in DMSO), or amiodarone plus AR3 and incubated for 4 days. Cultures were collected by filtration and the net dry weight of Aspergillus cells measured (Table 2). The ratio of cells treated with drug versus control cells receiving only DMSO was calculated (Table 2).

The mean ratio from the three experiments shown in Table 2 were determined, revealing a synergistic effect of the two drugs. 15 μM amiodarone treatment had no deleterious effect on growth of Aspergillus, possibly increasing growth slightly to 113% (std. error=18) of control. 25 μM AR3 showed a slight growth inhibition of 77% (std. error=14) of control. In striking contrast, the combination of AR3 plus amiodarone showed a marked growth inhibition of 3.7% (std. error=1.3), which was more than 20-fold more inhibitory than either drug by itself.

These results show that AR3 synergizes with amiodarone to have a major inhibitory effect on growth of Aspergillus. Such effects on Aspergillus represent a significant advance in the development of an effective antifungal treatment for this difficult pathogen.

TABLE 2
Net dry weight of cells (grams-% Growth)¹

| | Experiment 1 | Experiment 2 | Experiment 3 |
|---|---|---|---|
| | 20 mL | 20 mL | 5 mL |
| DMSO | 0.1091 (100) | 0.1422 (100) | 0.0330 (100) |
| Amiodarone | 0.1625 (149) | 0.1429 (100) | 0.0293 (89) |
| AR3 | 0.1093 (100) | 0.0744 (52) | 0.0260 (79) |
| AR3 + Amiodarone | 0.0011 (1) | 0.0068 (5) | 0.0017 (5) |

¹% Growth is the ratio of cell growth for each treatment versus treatment with the carrier, DMSO, only. Thus, DMSO is given a value of 100%. Additions: DMSO, 30 μl; Amiodarone, 15 μM; AR3, 25 μM.

### REFERENCES

Agrios, G. N. (1997) Plant Pathology. San Diego, Calif.: Academic Press.
Bastert, J., Schaller, M., Korting, H. C., and Evans, E. G. V. (2001) Current and future approaches to antimycotic treatment in the era of resistant fungi and immunocompromised hosts. Internat. J. of Antimicrob. Agents 17: 81-91.
Batiza, A. F., Schulz, T., and Masson, P. H. (1996) Yeast respond to hypotonic shock with a calcium pulse. J Biol Chem 271: 23357-23362.
BeDell (1996) Amiodarone. In Physicians GenRx. BeDell, e.a. (ed). St. Louis, Mo.
Bodey, G., Bueltmann, B., Duguid, W., Gibbs, D., Hanak, H., Hotchi, M., Mall, G., Martino, P., Meunier, F., Milliken, S., and et al. (1992) Fungal infections in cancer patients: an international autopsy survey. Eur J Clin Microbiol Infect Dis 11: 99-109.
Casadevall, A., and Perfect, J. R. (1998) Cryptococcus neoformans. Washington, D.C.: American Society for Microbiology Press.
Courchesne, W. E. (2002) Characterization of a novel, broad-based fungicidal activity for the antiarrhythmic drug amiodarone. Journal of Pharmacology and Experimental Therapeutics 300: 195-199.
Courchesne, W. E., and Ozturk, S. (2003) Amiodarone induces a caffeine-inhibited, MID1-depedent rise in free cytoplasmic calcium in Saccharomyces cerevisiae. Mol Microbiol 47: 223-234.
DiDomenico, B. (1999) Novel antifungal drugs. Cur. Opin. Microbiol. 2: 509-515.
Fostel, J. M., and Lartey, P. A. (2000) Emerging novel antifungal agents. Drug Discov Today 5: 25-32.
Georgopapadakou, N. H., and Walsh, T. J. (1996) Antifungal agents: chemotherapeutic targets and immunologic strategies. Antimicrob Agents Chemother 40: 279-291.
Gill, J., Heel, R., and Fitton, A. (1992) Amiodarone, An Overview of its Pharmacological Properties, and Review of its Therapeutic Use in Cardiac Arrhythmias. Drugs 43: 69-110.
Graybill, J. (1996) The Future of Antifungal Therapy. Clinical Infectious Diseases 22(Suppl 2): S166-S178.
Groll, A. H., and Walsh, T. J. (2001) Uncommon opportunistic fungi: new nosocomial threats. Clin Microbiol Infect 7 Suppl 2: 8-24.
Hawksworth, D. L. (2001) The magnitude of fungal diversity: the 1.5 million species estimate revisited. Mycological Research 105: 1422-1432.
Jones, J. L., Hanson, D. L., Dworkin, M. S., Alderton, D. L., Fleming, P. L., Kaplan, J. E., and Ward, J. (1999) Surveillance for AIDS-defining opportunistic illnesses, 1992-1997. MMWR 48: 1-22.
Kanbe, T., Han, Y., Redgrave, B., Riesselman, M. H., and Cutler, J. E. (1993) Evidence that mannans of Candida albicans are responsible for adherence of yeast forms to spleen and lymph node tissue. Infect Immun 61: 2578-2584.
Klotz, S. A., and Smith, R. L. (1992) Glycosaminoglycans inhibit Candida albicans adherence to extracellular matrix proteins. FEMS Microbiol Lett 78: 205-208.
Kossakowski J., Ostrowska, K. Hejchman, E. and Wolska, I., (2005) Synthesis and structural characterization of derivatives of 2- and 3-benzo[b]furan carboxylic acids with potential cytotoxic activity. II Farmaco 60:519-527.
Kwon-Chung, K. J., and Bennett, J. E. (1992) “Cryptococcosis”. In Medical Mycology. Lea and Febiger (eds). Philadelphia, pp. 397-446.
Kwon-Chung, K. J., Sorrell, T. C., Dromer, F., Fung, E., and Levitz, S. M. (2000) Cryptococcosis: clinical and biological aspects. Med Mycol 38 Suppl 1: 205-213.
Marr, K. A., Carter, R. A., Crippa, F., Wald, A., and Corey, L. (2002) Epidemiology and outcome of mould infections in hematopoietic stem cell transplant recipients. Clin Infect Dis 34: 909-917.
Mason, J. W. (1987) Amiodarone. New England Journal of Medicine 316: 455-466.
Mitchell, T. G., and Perfect, J. R. (1995) Cryptococcosis in the era of AIDS-100 years after the discovery of Cryptococcus neoformans. Clin. Microbiol. Rev. 8: 515-548.
National Nosocomial Infections Surveillance, N., System Report (1999) Data summary from January 1990-May 1999, issued June 1999. Am J Infect Control 27: 520-532.
Pfaller, M. A., Jones, R. N., Messer, S. A., Edmond, M. B., and Wenzel, R. P. (1998) National surveillance of nosocomial blood stream infection due to Candida albicans: frequency of occurrence and antifungal susceptibility in the SCOPE Program. Diagn Microbiol Infect Dis 31: 327-332.
Quaglino, D., Huy Riem Ha, Elena Duner, Daniela Bruttomesso, Laurent Bigler, Ferenc Follath, Giuseppe Realdi, Andrea Pettenazzo, and Aldo Baritussio (2004) Effects of metabolites and analogs of amiodarone on alveolar macrophages: structure-activity relationship. Am J Physiol Lung Cell Mol Physiol 287: L438-L447,
Roden, D. M. (1996) Ionic mechanisms for prolongation of refractoriness and their proarrhythmic and antiarrhythmic correlates. Am. J. Cardiol. 78: 12-16.
Salvi, V. S., Sethna, S.(1967) Studies in Furan Derivatives; Part II. Synthesis of some bromobenzofuran derivatives. J. Indian Chem. Soc.
Singh, B. N. (1996) Antiarrhythmic actions of amiodarone: a profile of a paradoxical agent. Am. J. Cardiol. 78: 41-53.
Singh, N., and Paterson, D. L. (2005) Aspergillus infections in transplant recipients. Clin Microbiol Rev 18: 44-69.
Stansell, J. D. (1993) Pulmonary fungal infections in HIV-infected persons. Semin. Respir. Infect. 8: 116-123.
Sugar, A. M. (1991) Overview: Cryptococcosis In The Patient With AIDS. Mycopathologia 114: 153-157.
Sussman, A., Huss, K., Chio, L. C., Heidler, S., Shaw, M., Ma, D., Zhu, G., Campbell, R. M., Park, T. S., Kulanthaivel, P., Scott, J. E., Carpenter, J. W., Strege, M. A., Belvo, M. D., Swartling, J. R., Fischl, A., Yeh, W. K., Shih, C., and Ye, X. S. (2004) Discovery of Cercosporamide, a known antifungal natural product, as a selective Pkc1 kinase inhibitor through high-throughput screening. Eukaryot Cell 3: 932-943.
USPDI (1997) Amiodarone. In Drug Information for the HealthCare Profession. Twinbrook Parkway, Md., pp. 80-83.

1. A compound of formula: 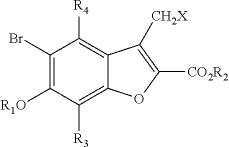
or a pharmaceutically acceptable salt thereof,
wherein R₁, R₂ and R₄ are H, or an alkyl having 1-6 carbon atoms where the alkyl group is optionally substituted with one or more halogens, hydroxyl groups, amino groups or a combination thereof;
R₃ is selected from the group consisting of H, alkyl, —CO—R, —OCO—R, —NR′CO—R, —CO—N(R)₂, and —CO₂—R, where R and R′ are independently selected from H or an alkyl group having from 1 to 6 carbon atoms, wherein the alkyl group is optionally substituted with one or more halogens, OH, amino groups or a combination thereof; and
X is selected from the group consisting of H, halogen, OH, alkylsulfonate, arylsulfonate, and an amino group with the exceptions that R₃ and R₄ are not both H and that R₃ is a group other than H when X is Br.

2-7. (canceled)

8. The compound of claim 1 wherein R₁ is hydrogen.

9. The compound of claim 1 wherein R₂ is hydrogen or an alkyl group.

10. The compound of claim 1 wherein R₂ is a methyl, ethyl or propyl group.

11. The compound of claim 1 wherein R₃ is a —CO—R group.

12. The compound of claim 1 wherein R₃ is a —CO—CH₃ group.

13. The compound of claim 1 wherein R₃ is a —CO—R group, and R₄ is hydrogen or a methyl group.

14. The compound of claim 13 wherein X is Br.

15. The compound of claim 14 wherein R₁ is H.

16. The compound of claim 15 wherein R₂ is a methyl group.

17. The compound of claim 1 wherein R₁ and R₄ are H, R₂ is a methyl group and R₃ is —CO—CH₃.

18. A pharmaceutical composition comprising a pharmaceutically acceptable excipient or carrier and one or more compounds of claim 1 in a therapeutically effective amount or a combined therapeutically effective amount for treatment of a fungal infection.

19. A pharmaceutical composition comprising a pharmaceutically acceptable excipient or carrier and an amiodarone compound in combination with one or more compounds of claim 1 in a combined therapeutically effective amount for treatment of a fungal infection.

20. The pharmaceutical composition of claim 19 wherein the amiodarone compound is amiodarone or a pharmaceutically acceptable salt thereof.

21-22. (canceled)

23. A method of treating a fungal infection comprising administering to an individual suffering from the fungal infection a therapeutically effective amount or a combined therapeutically effect amount of one or more compounds of claim 1.

24. A method of treating a fungal infection comprising administering to the individual suffering from the fungal infection a combined therapeutically effective amount of an amiodarone compound and one or more compounds of claim 1.

25-26. (canceled)

27. A method of killing or inhibiting replication of fungi comprising contacting the fungi with one or more compounds of claim 1 in an amount or a combined amount effective for killing or inhibiting replication of the fungi.

28. A method of killing or inhibiting replication of fungi comprising contacting the fungi with one or more compounds of claim 1 and an amiodarone compound in a combined amount effective for killing or inhibiting replication of the fungi.

29. An agricultural composition for application to plants for the prevention or treatment of fungal infections and infestations which comprises one or more compounds of formula I in an amount or combined amount effective for preventing or treating a fungal infection of a plant by a at least on plant pathogenic fungus.
